(19) United States
(12) Patent Application Publication
(10) Pub. No.: US 2008/0188860 A1
(43) Pub. Date: Aug. 7, 2008

(54) Ophthalmic surgical apparatus

(76) Inventor: Steven D. Vold, Bentonville, AR (US)

Correspondence Address: TROP PRUNER & HU, PC, 1616 S. VOSS ROAD, SUITE 750, HOUSTON, TX, 77057-2631, US

(21) Appl. No.: 12/012,877

(22) Filed: Feb. 6, 2008

Related U.S. Application Data
(60) Provisional application No. 60/900,041, filed on Feb. 7, 2007.

(51) Int. Cl. A61F 9/007; A61B 1/32

(52) U.S. Cl. 606/107; 600/217

(57) ABSTRACT

In one embodiment, an ophthalmic surgical apparatus is provided for retracting the capsule. The device includes a hook member that is in the conduit in a nondeployed state. However, after inserting a portion of the conduit into an incision, the hook member may be slid out from the conduit to then retract the capsule tissue against the distal end of the conduit.

This application claims priority to U.S. Provisional Patent Application No. 60/900,041 filed on Feb. 7, 2007 entitled OPHTHALMIC SURGICAL APPARATUS.

### BACKGROUND

FIG. 1 is a general representation of a portion of the human eye. The lens 131 is enclosed within a capsule 120. The iris 115 is located between the posterior chamber 135 and the anterior chamber 110 of the eye. The cornea 105 is adjacent to the anterior chamber 110. The zonule of Zinn (zonule) 125 is a ring of fibrous strands connecting the ciliary body 140 with the lens 130. The zonular fibers hold the lens in place and help the eye focus by contracting and relaxing according to the desired range of focus.

If a patient suffers from weakened zonule fibers, complications may arise in ophthalmic surgery. For example, for cataract removal the surgeon makes a small incision in the capsule 120 before attempting to replace the lens 130 with, for example, a synthetic intraocular lens. However, in a patient with weak zonular support, the support for the replacement lens is compromised. In such cases, a surgeon may use a capsular tension ring (CTR) to compensate for the poor zonular support. Still, CTRs are generally used in cases of mild, generalized zonular weakness and may not suffice for patients with more advanced zonular weakness. Furthermore, CTRs are not ideal when the patient has an anterior capsular tear, a discontinuous capsulorhexis, or a posterior capsular tear.

As another option for these more complicated scenarios, the surgeon may use one or more capsular retractors to “hook” or hold the capsule in place while accessing the cataract and implanting a replacement lens. The retractor may then function as an artificial zonule. Unfortunately, due to the retractor's focused, concentrated pressure, the retractor may cause a capsular tear and/or retractor dislodgement, which may lead to complications such as glaucoma and/or instability for the patient's lens 6r a replacement lens. For example, a torn capsule may lead to a lens, natural or artificial, dropping below the retina.

### BRIEF DESCRIPTION OF THE DRAWINGS

The accompanying drawings, incorporated in and constituting a part of this specification, illustrate one or more implementations consistent with the principles of the invention and, together with the description of the invention, explain such implementations. The drawings are not necessarily to scale, the emphasis instead being placed upon illustrating the principles of the invention. In the drawings:

FIG. 1 is a general representation of the human eye.

FIG. 2 discloses an ophthalmic surgical apparatus retracting ocular tissue in one embodiment of the invention.

FIG. 3 discloses an ophthalmic surgical apparatus in one embodiment of the invention.

FIG. 4 discloses an ophthalmic surgical apparatus in one embodiment of the invention.

FIG. 5 discloses an ophthalmic surgical apparatus traversing an incision in ocular tissue in one embodiment of the invention.

FIG. 6 discloses an ophthalmic surgical apparatus retracting ocular tissue in one embodiment of the invention.

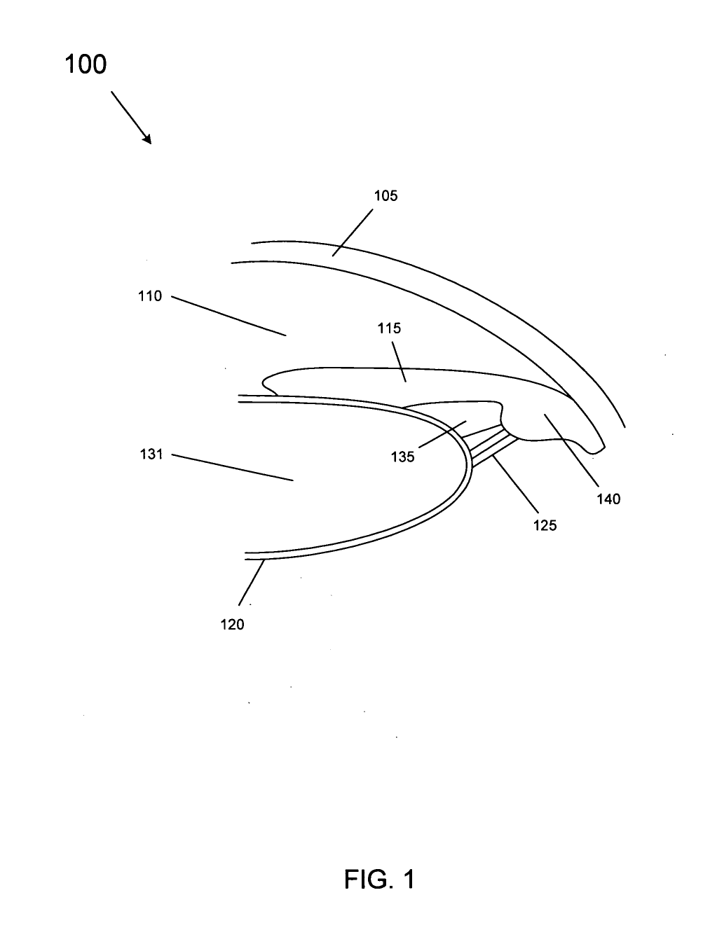

### DETAILED DESCRIPTION

The following description refers to the accompanying drawings. While the description provides a thorough understanding of the various aspects of the claimed invention by setting forth specific details such as particular structures, architectures, interfaces, and techniques, such details are provided for purposes of explanation and should not be viewed as limiting. Moreover, those of ordinary skill in the art will, in light of the present disclosure, appreciate that various aspects of the invention claimed may be practiced in other examples or implementations that depart from these specific details. At certain junctures in the following disclosure, descriptions of well known devices and methods have been omitted to avoid clouding the description of the present invention with unnecessary detail.

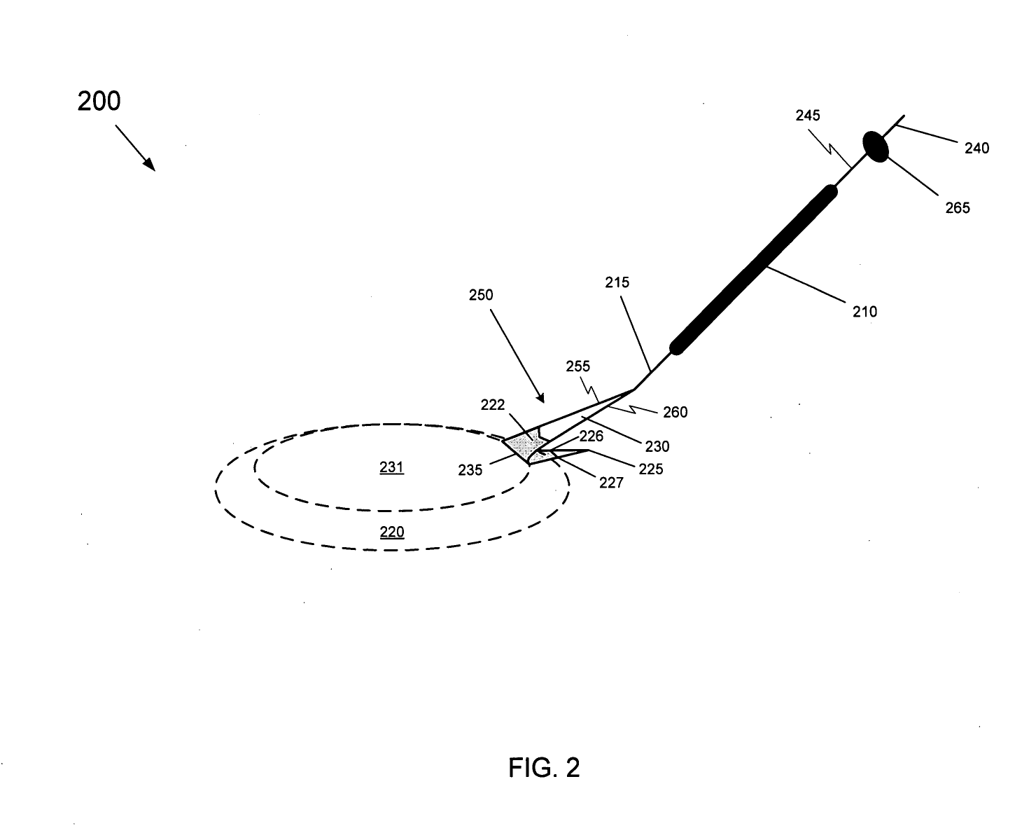

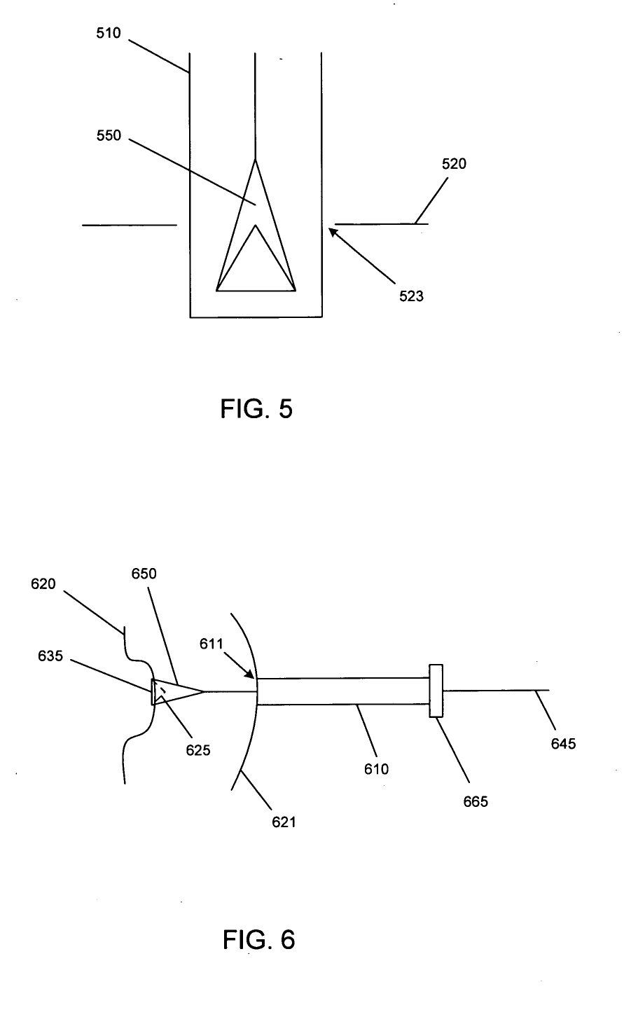

FIG. 2 discloses an ophthalmic surgical apparatus in one embodiment of the invention. The device includes a shaft 245, having a proximal portion 240 and a distal portion 215. A flexible hook 250 is connected to the shaft 245. The hook 250 includes a proximal portion 230 that is connected to the shaft 245. The hook 250 also includes a distal portion 235 which, in one embodiment of the invention, is wider than the proximal portion 230 of the hook. The hook 250 may include narrowing edges 226, 227 that join at a terminal point 225. The hook 250 may be deformable so that it can be located within a conduit 210, wherein the conduit 210 has a diameter that is less than the width of the distal portion 235 of the hook 250. For example, as further described in regards to FIG. 5, the hook 250 may be resilient, thereby allowing it to be deformed when fitted within the conduit 210. In one embodiment of the invention, the hook may include a membrane 222 located between two side hook members 255, 260. In other embodiments of the invention, the hook 250 may be of unitary construction. When the hook 250 has a broadened distal portion 235, retraction forces are evenly distributed and non-focused upon the ocular tissue, thereby lessening concentrated or narrowly focused forces that may traumatize the lens capsule or other ocular tissue. In one embodiment of the invention, the distal portion 235 is approximately 1 mm in width.

In one embodiment of the invention, as seen in FIG. 5, the surgeon utilizes the device by making a small incision (e.g., 1 mm) 523 in the capsule 520. With the hook 550 positioned within the conduit 510, the surgeon inserts the conduit 510 and hook 550 through or within the incision 523. Use of the conduit 510 in such a manner, with the hook positioned within the conduit, may help the surgeon accurately insert, place and manipulate the hook. The surgeon may use the conduit 510, with the hook 550 located within the conduit, to traverse the incision 523 in the capsule 520. However, in another embodiment of the invention, the surgeon may use the conduit, with the hook positioned within the conduit, to merely traverse an incision in, for example, an outer tissue in the eye (e.g., cornea). The surgeon may then deploy the hook from the conduit (as described below in relation to FIG. 2) before positioning the hook across or within the incision in an inner tissue, such as the capsule.

As seen in FIG. 2, after inserting the hook 250 into the eye, the surgeon slides the conduit 210 proximally to deploy the resilient hook 250 from the conduit. The surgeon positions the distal portion 235 of the hook adjacent to the incision in the capsule 220. The surgeon then retracts the capsule 220 using the hook 250 to expose the lens 231.

The retraction of ocular tissue (e.g., capsule tissue) may be maintained in a number of ways. As seen in FIG. 6, the retracted capsule 620 may be positioned between the distal portion 635 of the hook 650 and the distal portion 611 of the conduit 610. The conduit 610 is prevented from sliding proximally along the shaft 645 by a fixation device 665. The fixation device (e.g., silicon stopper) may be fixed to the shaft 645. However, it may also be slid along the shaft 645 with the requisite level of force. In other words, the hook 650 may be deployed into the eye and outside of the conduit by sliding the conduit 610 proximally along the shaft 645. The conduit 610 may then be slid distally along the shaft 645 to abut, for example, outer ocular tissue (e.g., sclera 621). Thus, the capsule 620 tissue is retracted or pinched in between the hook 650 and conduit 610. The conduit is held in place by, for example, the ocular tissue (e.g., sclera 621) and the fixation device 665. The degree of retraction may be adjusted by, for example, pulling the shaft 645 proximally to produce slack between the fixation device 665 and conduit 610. The fixation device 665 may then be slid distally along the shaft 645 to abut the proximal end of the conduit 610, thereby taking up the slack and maintaining the increased level of retraction.

In another embodiment of the invention, the conduit may be held in place due to a broadened portion of the shaft. In other words, the cross-section of the proximal portion of the shaft may be larger than the cross-section of the distal portion of the shaft and the cross-section of the conduit. The broadening of the shaft may be graduated. Thus, the conduit is prevented from sliding proximally past a certain point where the shaft is wider than the conduit.

Again referring to FIG. 6, to withdraw the device from the eye, the surgeon slides the conduit 610 and fixation device 665 proximally along the shaft 645 to discontinue or lessen retraction of the capsule 620. The hook 650 may then be moved distally, toward the center of the eye, and out or away from the eye, until the terminal end 625 of the hook is removed from the eye. In one embodiment of the invention, the terminal end 625 of the hook is more flexible than the distal portion 635 of the hook 650, thereby facilitating removal of the apparatus from the eye, while still providing sufficient rigidity at the distal portion 635 to facilitate retraction of ocular tissues, such as the capsule or iris.

In one embodiment of the invention, the fixation device may be permanently connected to one end of the conduit. For example, the fixation device may be coupled at or near the proximal end of the conduit. The distal end of the conduit, along with the hook, may be inserted into the eye. The distal end may traverse an incision in the eye or, for example only, be positioned proximate but not across or within the incision. The conduit may then be slid proximally thereby deploying the hook, which may or may not be resilient. The conduit may be slid completely off the shaft. The physician may then flip or rotate the conduit and then slide the conduit back onto the shaft. The fixation member may now be located distally on the conduit. The physician may then retract tissue (e.g., capsule or iris) with the hook against the fixation device. In other words, in one embodiment of the invention the retracted tissue or tissues may be compressed immediately between the hook and the fixation device. During retraction, the conduit (primarily or entirely located proximal to the fixation device) would provide stiffness to a flexible shaft while the fixation device provides a larger surface area to press against an incision site, thereby facilitating stable retraction.

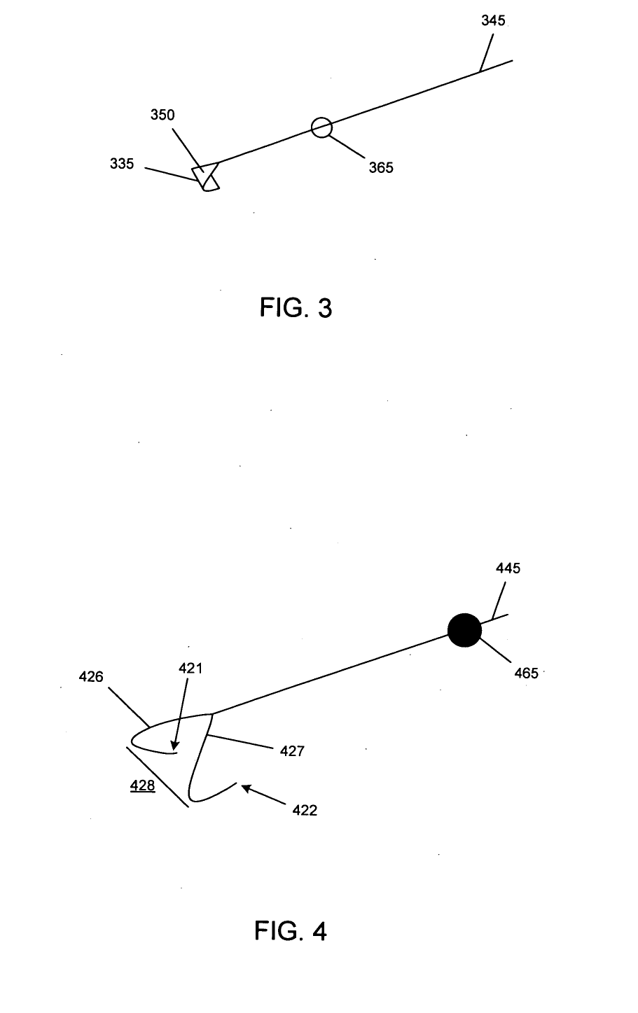

FIG. 3 shows an embodiment of the invention wherein the surgical apparatus includes a shaft 345 coupled to a hook 350, the hook 350 including a distal portion 335 having a width greater than 0.3 mm. The device may further include a fixation member 365 (e.g., silicon stopper) slidably coupled to the shaft to provide retraction force to ocular tissue (e.g., capsule). The ocular tissue may be held in a retracted state by positioning the tissue or tissues between the distal portion 335 of the hook and the fixation member 365. For example, the capsule may be retracted by positioning the capsule adjacent to the distal portion 335 of the hook, and positioning the sclera (or some other ocular tissue) adjacent to the fixation member 365.

FIG. 4 shows an embodiment of the invention wherein the surgical apparatus includes a shaft 445 coupled to hook members 426, 427, the hook members separated from one another by distance 428. The distance 428 may be considerably broader than the diameter or width of shaft 445. The distance 428 may be at least 0.3 mm. The tips 421, 422 of the hook members may be angled away from one another, on divergent paths, in one embodiment of the invention. The device may further include a fixation member 465 (e.g., silicon stopper) slidably coupled to the shaft to provide retraction force to ocular tissue. The ocular tissue may be held in a retracted state, positioned between the distal portion of the hook members 426, 427 and the fixation member 465. In one embodiment of the invention, a conduit, as described above, may be used to add stiffness to the device, wherein the hook members are flexible. In a first position, the hook members 426, 427 may be located inside the conduit. The added rigidity from the conduit may facilitate insertion, placement, and manipulation of the device. The conduit may be moved proximally, into a second position wherein the hook members are no longer located within the conduit, after the device is deployed in the eye. In one embodiment of the invention, the conduit may be moved distally and repositioned over the flexible hook device to facilitate removal of the device.

In various embodiments of the invention, the hook 250 may be composed of, for example only, acrylic, nylon, plastic polymethyl methacrylate, polypropylene, and/or ethylene. The stopper 265 may be composed of, for example, silicon. It will be understood that the above embodiments may be suitable for non-human (e.g., canine) ocular tissues. The device may be disposable. It will be further understood that the functionality of the conduit may be achieved in various ways. For example, a rigid member (e.g., a shaft) may be slidably coupled to the device to add rigidity as described above. The rigid member may be coupled to the device shaft (e.g., 240) using loops, staples, an orifice or orifices within the rigid member, or any number of fixation mechanisms known to those of ordinary skill in the art. Furthermore, in one embodiment of the invention, the hook may be substantially inflexible, yet still be capable of being placed within a conduit for manipulation and placement of the hook.

In various embodiments of the invention, ophthalmic tissue or tissues may be retracted between a hook and the conduit. In such an embodiment, a fixation member may be located between the hook and the conduit. The fixation member may or may not directly contact tissue. In another embodiment, when ophthalmic tissue or tissues are retracted between a hook and the conduit, there may be no fixation embodiment between the tissue and the conduit. The conduit may or may not directly contact tissue.

While the present invention has been described with respect to a limited number of embodiments, those skilled in the art will appreciate numerous modifications and variations there from. It is intended that the appended claims cover all such modifications and variations that falls within the true spirit and scope of this present invention.

What is claimed is:

1. A method comprising:
providing an ophthalmic surgical apparatus comprising a shaft coupled to a first hook and slidably coupled to a conduit, the first hook being compressed and located substantially within the conduit;
traversing an incision in an ophthalmic tissue with a portion of the conduit and the compressed first hook being located substantially within the conduit;
slidably retracting the conduit along the shaft and away from the first hook to allow the first hook to decompress; and
retracting the ophthalmic tissue between the decompressed first hook and the conduit.

2. The method claim 1, further comprising:
providing the ophthalmic surgical apparatus comprising the shaft coupled to a second hook, the second hook being compressed and located substantially within the conduit;
traversing the incision in the ophthalmic tissue with the portion of the conduit and the compressed second hook being located substantially within the conduit;
slidably retracting the conduit along the shaft and away from the second hook thereby allowing the second hook to decompress; and
retracting the ophthalmic tissue between the decompressed second hook and the conduit while retracting the ophthalmic tissue between the decompressed first hook and the conduit.

3. The method of claim 2, further comprising creating the incision with a maximum width that is less than an additional maximum width between the first and second decompressed hooks.

4. The method of claim 1, further comprising retracting the ophthalmic tissue between the decompressed first hook and the conduit; wherein a distal portion of the conduit provides resistance for retraction by directly connecting to another ophthalmic tissue.

5. The method claim 4, further comprising:
slidably coupling a fixation member to the shaft; and
coupling the fixation member to a proximal portion of the conduit to stabilize the conduit.

6. The method of 1, further comprising:
slidably advancing the conduit along the shaft and over a portion of the deployed first hook to compress the decompressed first hook within the conduit; and
removing the surgical apparatus from the eye.

7. The method of claim 6, further comprising:
slidably advancing the conduit along the shaft and over the portion of the deployed first hook to compress the decompressed first hook within the conduit in an unfolded state;
wherein the first hook is compressed in a folded state during the step of traversing the incision in the ophthalmic tissue with the portion of the conduit.

8. The method of claim 1, further comprising:
traversing the incision in the ophthalmic tissue with a second end portion of the conduit to locate the second end portion proximate to the incision and distal to a first end portion of the conduit, the first end portion of the conduit coupled to a fixation member;
sliding the conduit off the shaft;
rotating the conduit;
sliding the conduit onto the shaft to locate the first end portion of the conduit proximate to the incision and distal to the second end portion of the eye;
retracting the ophthalmic tissue between the decompressed first hook and the fixation device.

9. An ophthalmic surgical apparatus comprising:
a shaft coupled to a first hook member and to slidably couple to a conduit; the first hook member to be received within the conduit in a nondeployed state and to be slidably removed from the conduit in a deployed state to retract an ophthalmic tissue between the conduit and the first hook member.

10. The apparatus of claim 9, wherein the shaft is coupled to a second hook member, the second hook member to be received within the conduit in a nondeployed state and to be slidably removed from the conduit in a deployed state to retract the ophthalmic tissue between the conduit and the first and second hook members.

11. The apparatus of claim 9, wherein the first hook member is resilient and compressed when received within the conduit in the nondeployed state and uncompressed when slidably removed from the conduit in the deployed state.

12. The apparatus of claim 10, wherein the first and second hook members are resilient and compressed when received within the conduit in the nondeployed state and uncompressed when slidably removed from the conduit in the deployed state.

13. The apparatus of claim 10, wherein a membrane is coupled to the first and second hook members.

14. The apparatus of claim 9, wherein the most distal portion of the first hook member is at least 3 mm from the most distal portion of the second hook member when the first and second hook members are removed from the conduit and in the deployed state.

15. The apparatus of claim 9, further comprising a fixation device to slidably couple to the shaft to stabilize the conduit during tissue retraction.

16. The apparatus of claim 9, further comprising a fixation device to couple to the conduit, the coupled fixation device and conduit to removably and slidably couple to the shaft;
wherein the fixation device is to couple to the ophthalmic tissue to provide resistance for retraction by the first hook member.

17. The apparatus of claim 9, wherein the first hook member is offset from the shaft and noncollinear with the shaft.

18. An ophthalmic surgical apparatus comprising:
a shaft coupled to a first tissue retracting member and to slidably couple to a conduit, the first tissue retracting member to be received within the conduit in a nondeployed state and to be slidably removed from the conduit in a nondeployed state to retract the ophthalmic tissue between a fixation member and the first tissue retracting member;
wherein the fixation member is to couple to the shaft.

19. The apparatus of claim 18, wherein the fixation member is to removably couple to the shaft.

20. The apparatus of claim 18, wherein the conduit is to removably couple to the shaft, the fixation member is to removably couple to the shaft, and the conduit is permanently coupled to the fixation member.
